(19) United States
(12) Patent Application Publication
(10) Pub. No.: US 2005/0158725 A1
(43) Pub. Date: Jul. 21, 2005

(54) Method of amplifying nucleic acid by electromagnetic induction heating and reaction container and reaction device to be used therein

(76) Inventors: Tetsuo Yukimasa, Nara (JP); Nobuhiko Ozaki, Ikoma (JP); Masaya Nakatani, Takaraduka (JP); Hidenobu Yaku, Kadoma (JP); Hiroaki Oka, Hirakata (JP); Kimi Fukahi, Osaka (JP)

Correspondence Address: WENDEROTH, LIND & PONACK, L.L.P., 2033 K STREET N. W., SUITE 800, WASHINGTON, DC, 20006-1021, US

(21) Appl. No.: 10/510,819

(22) PCT Filed: Sep. 24, 2003
(86) PCT No.: PCT/JP03/12122

(30) Foreign Application Priority Data
Sep. 24, 2002 (JP) 2002-277003

(51) Int. Cl. C12Q 1/68; C12M 1/34

(52) U.S. Cl. 435/6; 435/287.2

(57) ABSTRACT

The present invention provides a reaction container, a reaction device, and a method of amplifying a nucleic acid, which are suitable for locally heating a sample which contains a nucleic acid, enable rapid control of temperature changes in the sample, provide easy fabrication, and are low cost. The invention provides a reaction container used for a nucleic acid amplification reaction, which includes a sample holding portion formed from a cavity in the reaction container; and a heating element for heating a sample which contains a nucleic acid and which is held in the sample holding portion, wherein the heating element is made of a conductive material and arranged at a location where the sample or the sample and the periphery of the cavity is (are) to be locally heated, the conductive material generating heat by electromagnetic induction.

### TECHNICAL FIELD

The present invention relates to a method of amplifying a nucleic acid by electromagnetic induction heating, and a reaction container and a reaction device used in the method. In particular, the present invention relates to a method of amplifying a nucleic acid using a polymerase chain reaction by electromagnetic induction heating, and to a reaction container and a reaction device used in the method.

### BACKGROUND ART

In recent years, techniques associated with gene information have been actively developed. In the medical field, by analyzing disease-related genes, the treatment of diseases at a molecular level is becoming possible. In addition, by genetic diagnosis, tailor-made medical treatments that best suit each individual patient's needs are becoming possible. In the pharmaceutical field, gene information is used to identify protein molecules, such as antibodies and hormones, and the identified protein molecules are used as chemicals. In the agricultural and food fields too, many products are produced with the use of gene information.

In such techniques associated with gene information, one of the most important methods is a nucleic acid amplification reaction. Above all, a polymerase chain reaction method is a technique for amplifying only a specific part of a gene to millions of copies and is used in a wide variety of fields including medical microbiology, clinical diagnosis of inherited diseases, forensic medicine, and the like, as well as in research applications such as molecular biology. In particular, in genetic diagnosis techniques in clinical practice, faster analysis is demanded. In the polymerase chain reaction method too, development of techniques for faster polymerase chain reaction method is demanded.

Amplification of a gene by the polymerase chain reaction method is performed by repeating three steps regarded as one cycle, for 30 to 35 cycles; the steps includes separation (thermal denaturation) of double-stranded DNA into single-stranded DNA, binding (annealing) of primers, and extension (extension reaction) of DNA by polymerase. Although the steps may differ depending on conditions, normally, each step is performed under the following conditions: the thermal denaturation is 94° C.×1 minute; the annealing is 50-60° C.×1 minute; and the extension reaction is 72° C.×1-5 minutes (for example, see Japanese Laid-Open Patent Publication No. 62-000281).

Generally, in a PCR device, the time required for one complete cycle of temperature change in a PCR reaction is considered to be the sum of: (1) the time it takes for an aluminum block for heating and a reaction container or the like for holding a sample to reach the target temperature; (2) the time it takes for a sample solution to uniformly reach the target temperature; and (3) the time it takes for denaturation, annealing, and strand extension reactions themselves to complete. It is considered that the time (1) mainly depends on the heat capacity, size, or the like of the aluminum block, reaction container, or the like and the time (2) mainly depends on sample volume, the shape of the container, or the like. As described above, in the polymerase chain reaction, it is required to repeat a temperature change within a range of the order of 40° C. 30 times or more. In conventional devices for performing the polymerase chain reaction, the temperature is raised by putting a sample in a polypropylene tube and using an aluminum block having a large heat capacity, and thus heating or cooling takes time; accordingly, the polymerase chain reaction requires several hours or more to complete.

Meanwhile, there has been an attempt to reduce the time required for the polymerase chain reaction by reducing the size of such a polymerase chain reaction device. For example, there has been a report of microfabricated PCR reaction vessels which are integrated on a CE (capillary electrophoresis) chip (Japanese Laid-Open Patent Publication No. 2000-201681). The integrated PCR-CE device includes: a cavity which is provided by performing chemical etching between two glass substrates; and a resistance heater provided by performing metal thin film deposition on the inner or outer surface of the cavity to heat a sample in the cavity. The above publication describes that according to the PCR-CE device, in the case of performing PCR thermocycling, the heating rate is 20° C./second and the cooling rate is 2° C./second, with a reaction volume of less than 1/10 μl. The PCR-CE integrated device requires about 60 seconds to complete one heating-cooling cycle, which means that performing 30 cycles takes about 30 minutes.

As an example in which thermocycling time is further reduced, there has been a report of a microfabricated device for PCR which includes: a chip-like glass microchamber for holding a sample; a tungsten lamp which serves as an infrared radiation source for non-contact heating; and a solenoid-gated compressed air source for cooling (“Infrared-Mediated Thermocycling for Ultrafast Polymerase Chain Reaction Amplification of DNA”, Oda et al., “Analytical Chemistry”, U.S.A., Oct. 15, 1998, Vol. 70, No. 20, pp. 4361-4368). In this device, a sample is heated with the infrared radiation source arranged with a distance of about 2 cm from the glass microchamber, and the sample is cooled using compressed air at room temperature, whereby one heating-cooling cycle for the sample with a volume of the order of 5 μl can be completed in approximately 17 seconds.

Further, as an example in which the heating-cooling cycle time is reduced, a micro device has been developed which enables rapid thermocycling by locally heating a solution using an infrared (IR) laser (“Photothermal Temperature Control of a Chemical Reaction on a Microchip Using an infrared Diode Laser”, Slyadnev et al., “Analytical Chemistry”, U.S.A., Aug. 15, 2001, Vol. 73, No. 16, pp. 4037-4044). According to this device, a heating rate of 67° C./second and a cooling rate of 53° C./second are possible using a sample with a volume of only 5 nl. This is equivalent to 30 times as rapid as the conventional system using a heating block and 3-6 times as rapid as the above-described electric-heating type microfabricated device.

### DISCLOSURE OF THE INVENTION

In the above-described conventional art, a reduction in the heating-cooling cycle for PCR is achieved by reducing a reaction volume to the order of several μl to several nl or further using a non-contact type heater, so as to reduce the heat capacity of the device or to minimize the influence of the heat capacity exerted on the device. These devices, however, still leave problems described below.

For example, in the case of the PCR-CE chip of Japanese Laid-Open Patent Publication No. 2000-201681, the resistance heater for heating a sample is deposited on the PCR-CE chip by metal thin film deposition and used; in this case, some problems may arise such as poor adhesion to the substrate which may be caused depending on the types of metal thin film and difficulty in heating due to an elevated resistance caused by oxidation during a repetition of thermocycling. Further, in the case where the heater is arranged within the cavity, when the cavity is heated to 95° C., degradation of the coating of the side wall of the heater is caused, resulting in a loss of electrical continuity. In order to overcome these problems, in Japanese Laid-Open Patent Publication No. 2000-201681, platinum/titanium is used as a metal coating and further the heater is deposited on an external flat surface of the chip; however, there is a description that arranging the heater on the external flat surface of the chip causes a delay in temperature in the cavity. In any case, it is clear that there are many limitations in the use of such a resistance heater and thus fabrication of the device is not easy. In particular, in the case of using such a resistance heater, electric leads for applying a voltage to the heater to heat the reaction container are required but wiring for the electric leads is not easy and thus chip design becomes complex.

Moreover, in the example of using a tungsten lamp such as that of the above-described Oda et al., since the entire chip is heated, there is a problem that local heating cannot be performed such that only a cavity portion to be used for a PCR reaction is heated using a PCR-CE integrated chip such as that of the above-described Japanese Laid-Open Patent Publication No. 2000-201681, for example.

In addition, in the example of using an IR laser such as that of the above-described Slyadnev et al. (2001), a device which allows for a very high output as a heat source is required, resulting in a high cost of facilities.

Therefore, there is a need for a PCR device which accelerates a heating-cooling cycle, enables local heating, is easy to fabricate, and is low cost.

In view of the foregoing problems, it is an object of the present invention to provide a method of amplifying a nucleic acid which enables rapid amplification of DNA in a sample, is suitable for locally heating the sample, provides easy fabrication, and is low cost, and a reaction container and a reaction device used in the method. Further, it is another object of the present invention to provide a card-type PCR reaction device or a card-type diagnosis device used for the purpose of diagnosis.

In order to overcome the foregoing problems, the present invention provides a reaction container used for a nucleic acid amplification reaction. The reaction container comprises: a sample holding portion formed from a cavity in the reaction container; and a heating element for heating a sample which contains a nucleic acid and which is held in the sample holding portion, wherein the heating element is made of a conductive material and arranged at a location where the sample or the sample and a periphery of the cavity is (are) to be locally heated, the conductive material generating heat by electromagnetic induction.

Preferably, in the reaction container of the present invention, an opening portion for injecting or discharging the sample into or from the cavity may be provided. Depending on the need, the number of the opening portion may be two or more.

Preferably, the heating element may be a metal thin film.

Preferably, the heating element may compose a part of an inner wall of the container which defines the cavity, and when the sample is introduced into the cavity, the heating element and the sample may come into direct contact with each other.

Preferably, the heating element may compose a part of an inner wall of the container which defines the cavity, and may be coated with a polymer film so as to avoid direct contact with the sample when the sample is introduced into the cavity. Preferably, the polymer film may be made of a compound selected from the group consisting of a fluoro compound and a silicon compound. More preferably, a thickness of the polymer film may be in a range of 10 nm to 10 μm.

Preferably, the maximum volume of the cavity may be in a range of 1 μl to 1 ml.

Preferably, the nucleic acid amplification reaction may be a polymerase chain reaction.

In another aspect of the present invention, there is provided a nucleic acid amplification device. The nucleic acid amplification device comprises: the above-described reaction container; and a heating coil for causing the heating element to generate heat by electromagnetic induction.

Preferably, the nucleic acid amplification device may further comprise an alternating-current power supply for supplying a current to the heating coil.

Preferably, the nucleic acid amplification device of the present invention may further comprise a control portion for controlling on/off of the alternating-current power supply so that a heating and cooling cycle of the sample can be performed continuously.

In still another aspect of the present invention, there is provided a nucleic acid detection device. The nucleic acid detection device comprises: a substrate having provided therein a plurality of cavities, for holding a sample which contains a nucleic acid; and a heating element made of a conductive material which generates heat by electromagnetic induction, wherein the cavities are connected to each other by channels so that the sample can move between the cavities, and the heating element is arranged in the substrate so that the sample which is present in a specific cavity selected from the plurality of cavities can be selectively heated.

In yet another aspect of the present invention, there is provided a method of causing a nucleic acid amplification reaction by heating and cooling a sample which contains a nucleic acid. The method comprises: step (a) of locally heating the sample or the sample and a periphery of the sample by electromagnetic induction heating; and step (b) of cooling the sample, wherein the steps (a) and (b) are repeated twice or more.

According to the present invention, a nucleic acid amplification reaction which enables rapid amplification of DNA in a sample, and a reaction container and a reaction device which are used for the nucleic acid amplification reaction can be provided. Further, a nucleic acid detection device can be provided which is capable of amplifying a nucleic acid rapidly and detecting the amplified nucleic acid.

### BRIEF DESCRIPTION OF THE DRAWINGS

FIG. 1 is an exploded perspective view showing a structure of a reaction container according to an embodiment of the present invention.

FIG. 2 is a perspective view of the reaction container as viewed from the substrate side.

FIG. 3 is a perspective view showing a structure of a variant of the reaction container.

FIG. 4 is an exploded perspective view showing a structure of another variant of the reaction container.

FIG. 5 is a cross-sectional view showing a structure of still another variant of the reaction container.

FIGS. 6(a) and 6(b) are respectively a top view and a cross-sectional view in a longitudinal direction, showing a structure of yet another variant of the reaction container.

FIG. 7 is a schematic diagram showing a reaction device according to the embodiment of the present invention.

FIG. 8 is an electrophoresis picture showing the results of amplification products obtained when a polymerase chain reaction is performed using a reaction container of an example of the present invention and a reaction container of a comparative example of the present invention.

FIG. 9 is an electrophoresis picture showing the results of amplification products obtained when a polymerase chain reaction is performed using a reaction container of an example of the present invention and a reaction container of a comparative example of the present invention.

### BEST MODE FOR CARRYING OUT THE INVENTION

The embodiment of the present invention will be described below.

A reaction container according to an embodiment of the present invention which is used for a nucleic acid amplification reaction is characterized in that the reaction container includes: a sample holding portion formed from a cavity in the reaction container; and a heating element for heating a sample which contains a nucleic acid and which is held in the sample holding portion, in which the heating element is made of a conductive material which generates heat by electromagnetic induction and arranged at a location where the sample or the sample and the periphery of the cavity is(are) to be locally heated.

In addition, a nucleic acid amplification device according to the embodiment of the present invention is characterized in that the device includes: the above-described reaction container of the present invention; and a heating coil for causing the heating element to generate heat by electromagnetic induction.

Moreover, a nucleic acid detection device according to the embodiment of the present invention is characterized in that the device includes: a substrate having a plurality of cavities provided therein to hold a sample which contains a nucleic acid; and a heating element made of a conductive material which generates heat by electromagnetic induction, in which the cavities are connected to each other by channels so that the sample can move between the cavities, and the heating element is arranged in the substrate so that the sample which is present in a specific cavity selected from the plurality of cavities can be selectively heated.

Furthermore, a method of causing a nucleic acid amplification reaction by heating and cooling a sample which contains a nucleic acid, according to the embodiment of the present invention is characterized in that the method includes: step (a) of locally heating the sample or the sample and the periphery of the sample by electromagnetic induction heating; and step (b) of cooling the sample, in which the steps (a) and (b) are repeated twice or more.

With the above-described configuration, only a desired portion to be heated by electromagnetic induction is allowed to directly generate heat and thus the temperature rises extremely rapidly. In addition, since a portion of the reaction container other than the portion where the material which generates heat by electromagnetic induction is included is not heated, the temperature falls extremely rapidly due to the heat capacity of the reaction container. Accordingly, nucleic acid amplification can be done in a shorter time compared to a nucleic acid amplification reaction using a conventional reaction container made of an aluminum block, etc.

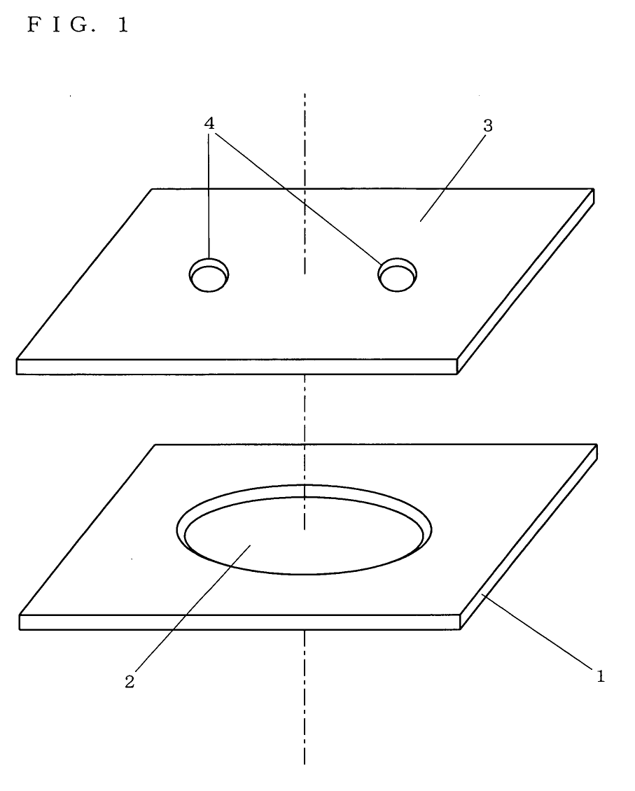

The embodiment of the present invention will be described in more detail below with reference to the drawings. FIG. 1 is an exploded perspective view showing a structure of a reaction container according to an embodiment of the present invention.

As shown in FIG. 1, a nucleic acid amplification reaction container of the present invention may be fabricated with a cavity 2 provided in a substrate 1 so that even a small amount of sample can be finely adjusted and that a rapid rise and fall in temperature can be achieved.

For the substrate 1 used for the nucleic acid amplification reaction container of the present invention, any material can be used as long as the material does not react with a sample solution; semiconductor such as silicon and germanium, glass such as silica glass, lead glass, and borosilicate glass, ceramic, or the like can be used. Further, it is also possible to use resins such as polystyrene (PS), polypropylene (PP), polyimide (PI), polytetrafluoroethylene (PTFE), polyphenylene sulfide (PPS), polyether ether ketone (PEEK), polyethylene terephthalate (PET), polyethylene naphthalate (PEN), and polymethyl methacrylate (PMMA). For a method of providing the cavity 2 in the substrate 1, in the case where the substrate 1 is a semiconductor such as silicon, dry etching such as RIE or wet etching using a strong alkaline etching solution or the like may be used, and in the case where the substrate 1 is a glass, wet etching using hydrofluoric acid or the like may be used. Above all, a semiconductor such as silicon is preferably used as the substrate 1 because with the semiconductor, a microcavity can be finely processed using microprocessing techniques which are known in the semiconductor field. In addition, in the case where the substrate 1 is a resin such as polystyrene, the cavity 2 may be provided using techniques such as molding, shaving, and imprinting. Further, the cavity 2 can also be provided in such a manner that a through-hole is provided in a sheet-like material by cutting and then the sheet-like material and a substrate are bonded together.

The cavity 2 thus provided is sealed with a cover plate 3 made of an appropriate material to prevent the sample from flowing out during a reaction. The material of the cover plate 3 may be the same as that of the substrate 1; however, it is preferred that the material of the cover plate 3 be completely bonded or adhered to the substrate 1 so as to seal the sample in the cavity 2. For example, in the case where the substrate 1 is a silicone and the cover plate 3 is a glass, sealing can be performed using techniques such as an anode bonding and a direct bonding. In addition, in the case where both the substrate 1 and the cover plate 3 are glasses, sealing can be performed by bonding using a technique of a hydrofluoric acid bonding or the like. Moreover, in the case where the substrate 1 and the substrate 3 are resins such as those described above, sealing can be performed using an adhesive or a heat seal.

The cover plate 3 has sample injection holes 4 provided therein to inject a sample into the cavity 2. At least one sample injection hole 4 should be provided, however, it is preferable to provide two or more sample injection holes 4 because with two or more sample injection holes 4, a sample injection hole 4 which is other than that used to inject the sample acts as a flow path of the air in the cavity 2, whereby the sample can be injected rapidly. After the injection of the sample, the sample injection holes 4 are sealed with a heat-resistant tape or the like, whereby the effect of preventing the sample from leaking or vaporizing from the sample injection holes 4 during a nucleic acid amplification reaction is provided.

The volume of the cavity may vary depending on the purpose; in general, the volume may range from several nl to several ml. For example, the maximum volume of the cavity may range from about 1 nl to about 10 nl, about 10 nl to about 100 nl, about 100 nl to about 1 μl, about 1 μl to about 10 μl, about 10 μl to about 100 μl, about 100 μl to about 1 ml, or about 1 ml to about 10 ml. Normally, the ideal volume of the cavity may range from the order of several μl to several ml. The shape of cavity is not limited to a circle and may be a polygon such as a triangle or a quadrangle.

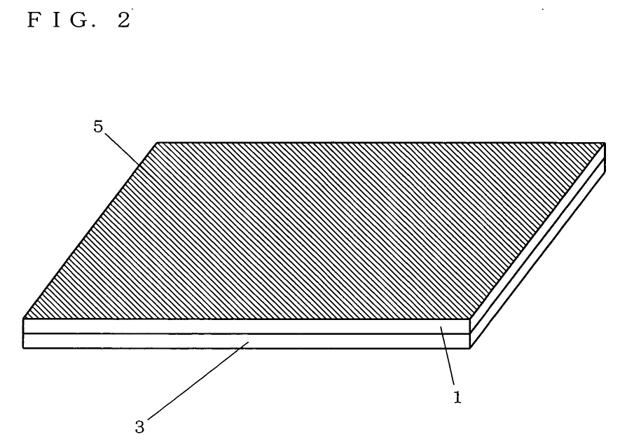

FIG. 2 is a perspective view of the reaction container of the present embodiment of the present invention, as viewed from the substrate side. A heat generating portion 5 containing a material which can generate heat by electromagnetic induction is provided on the entire backside (the side opposite to the side facing the cover plate 3) of the substrate 1.

A material which is contained in the heat generating portion 5 and can generate heat by electromagnetic induction includes stainless, iron, nickel, silver, copper, or aluminum. These metals may be used independently or in combination. Alternatively, an alloy or a clad metal which contains the above-described metals may be used.

The heat generating portion 5 may be attached in the form of a thin sheet, foil, or the like onto the substrate 1, or may be formed by depositing a film on the substrate 1 using the method of sputtering, vapor deposition, or the like.

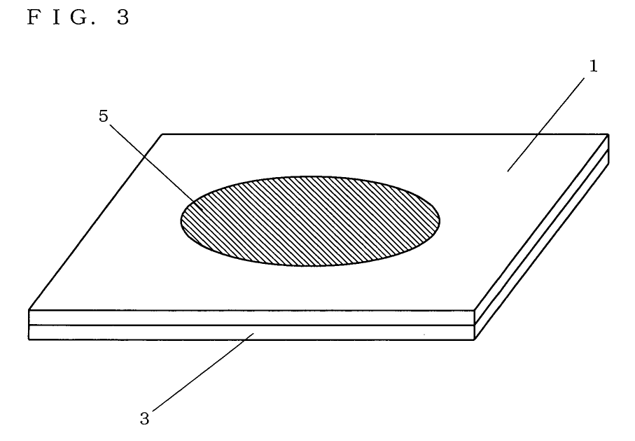

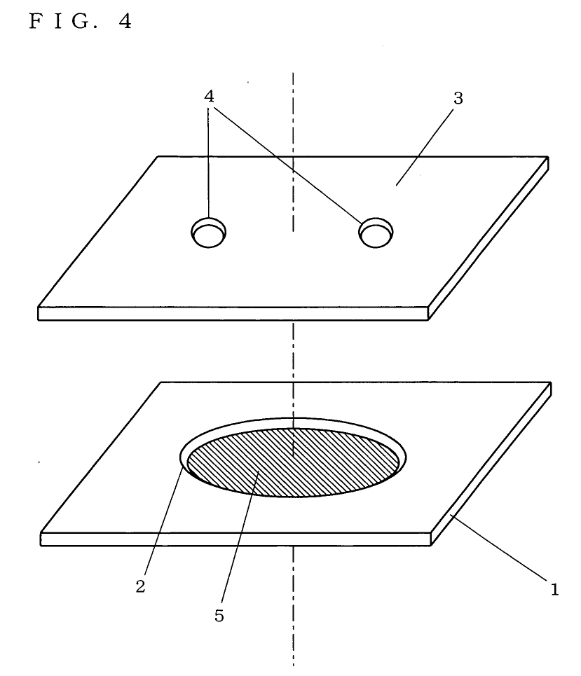

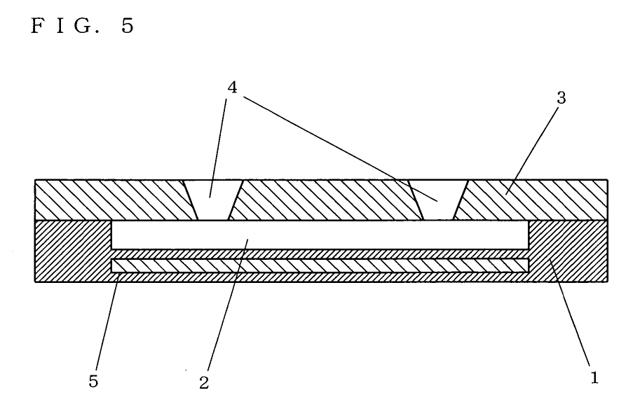

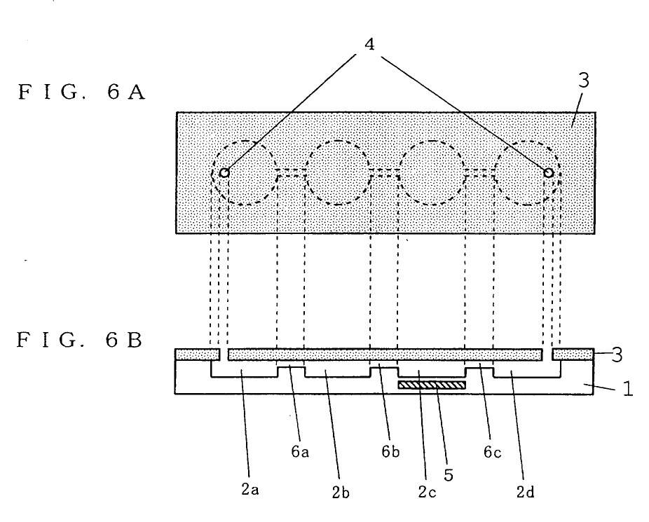

In FIGS. 3 to 6, there are shown the structures of variants of the reaction container according to the present embodiment of the present invention. FIG. 3 is a perspective view showing a structure of a variant of the reaction container, FIG. 4 is an exploded perspective view showing a structure of another variant of the reaction container, and FIG. 5 is a cross-sectional view showing a structure of still another variant of the reaction container. FIGS. 6(a) and 6(b) are respectively a top view and a cross-sectional view in a longitudinal direction, showing yet another variant of the reaction container of the present invention having a plurality of cavities.

In a variant, as shown in FIG. 3, a heat generating portion 5 is provided on the backside of a substrate 1 and on substantially an entire area where a cavity 2 is projected onto the backside of the substrate 1.

In another variant, as shown in FIG. 4, a heat generating portion 5 is provided in a cavity 2 and on substantially the entire bottom surface of the cavity 2. In this case, it is preferred that the heat generating portion 5 be formed at a location where bonding between a substrate 1 and a cover plate 3 is not hindered. In addition, it is preferred that at least a portion of the heat generating portion 5 with which a sample makes contact be made of a material which does not react with the sample. Such a material includes, for example, a polymer film such as a thin film containing a fluoro compound or a hydrophobic thin film containing a silicon compound. The thin film is not particularly limited to these materials, as long as the thin film does not react with a sample and can withstand a temperature rise of the order of 95° C. For the thickness of the thin film, the thinner the better in view of minimizing the possibility of heat diffusion; the film thickness is preferably about 10 nm to about 10 μm, more preferably about 10 nm to about 1 μm, most preferably about 10 nm to about 100 nm. The reason for a preferable film thickness of about 10 nm or more is that if the film thickness is smaller than 10 nm, there is a possibility of the heating element reacting with a sample.

For the thin film containing a fluoro compound, fluorocarbon may be used. A thin film of fluorocarbon can be formed in such a manner that a stainless sheet is placed in a fluorocarbon gas atmosphere, plasma is generated, and the stainless sheet is subjected to plasma processing. In addition, a thin film containing a fluoro compound can be formed by using a fluorinated silane coupling agent.

On the other hand, a thin film containing a silicon compound can be formed by a siliconizing process. As a siliconizing agent, for example, SIGMACOTE (registered trademark) (manufactured by Sigma Chemical Co.) may be used. A hydrophobic thin film can be formed such that a stainless sheet and a siliconizing agent are put in a desiccator and the air in the desiccator is evacuated, whereby the siliconizing agent is vaporized and the vapor from the silicon compound adheres onto the stainless sheet. In addition, a thin film can be formed by directly injecting a siliconizing agent onto a surface of a stainless sheet and then rinsing off the siliconizing agent with distilled water.

In still another variant, as shown in FIG. 5, a heat generating portion 5 is formed so as to be embedded in a substrate 1. In an exemplary method of forming a construction such as that shown in FIG. 5, the construction can be obtained by bonding a polymer film as a substrate 1. Here, as the polymer film, those using the above-described resin materials may be used; among these polymer films, a PET film is inexpensive and easily obtained and thus used most conveniently.

In FIG. 5, the shorter the distance between the heat generating portion 5 and a cavity 2 (i.e., the thickness of the polymer film), the better, in view of heating efficiency. Specifically, the upper limit of the distance is 1 mm or less, preferably 200 μm or less. In addition, the lower limit of the distance is 1 μm or more in view of the practical strength limitation of the polymer film, and preferably 5 μm or more.

In these variants, since the heating generating portion 5 is provided only in the vicinity of the cavity 2, these variants are less susceptible to the influence of the heat capacity of the substrate 1, achieving more rapid sample temperature rise and fall. In addition, as described above, the heating generating portion 5 of the present invention can be formed simply by attaching a heat generating material in the form of a thin sheet, foil, or the like onto the substrate 1 or by depositing a film on the substrate 1 using the method of sputtering, vapor deposition, plating, or the like. Accordingly, since it is not necessary to provide wiring or a heater terminal, an advantage is provided that the fabrication of a reaction container is easier than in the case where a resistance heater is used, for example. Further, because of the properties of the heat generating portion 5, the location, form, size, or the like of the heat generating portion 5 can also be easily changed depending on the purpose. To increase the heating efficiency of the sample, the heat generating portion 5 preferably has such a form that makes the projected area with respect to the cavity as great as possible.

The substrate may include a material which generates heat by electromagnetic induction. Doing so eliminates the need to additionally provide a heat generating portion, making the construction simple. In this case, as in the above-described case, it is preferred that at least a portion of the substrate with which a sample makes contact be made of a material which does not react with the sample.

In FIG. 6, as an application example of the above-described present invention, there is shown a representative embodiment of a card (or chip) type PCR device having a plurality of cavities (cavities 2a to 2d) which are arranged linearly and fluidly coupled to each other by channels 6a to 6c. In the device of FIG. 6, the cavity 2a is used to break down a cell and extracts genomic DNA. The cavity 2b is used to purify the extracted genomic DNA and the cavity 2c is used to amplify the purified DNA by a PCR reaction. The cavity 2d is used to detect the amplified DNA.

In this embodiment, the sample may be blood, saliva, hair root, or the like. The card-type device of the present invention may be used mainly for diagnosis purposes in hospitals or the like. Since the extraction, purification, amplification by PCR, and detection of genomic DNA from a cell in a sample can be done rapidly in a series of steps, the card-type device of the present invention may be particularly useful for the application of the Point of Care. Depending on reagents to be used, a reagent necessary for each cavity may be provided in the cavities in advance or immediately before use. The steps of extraction→purification→PCR of DNA may be integrally performed in one cavity.

The extraction of genomic DNA in the cavity 2a and the purification of genomic DNA in the cavity 2b may be performed using appropriate reagents or the like. For example, a reagent for breaking down a cell such as a leukocyte, an enzymatic reagent such as proteinase, or the like may be used. As a representative method for DNA extraction and purification, there is a method shown in “Rapid Isolation of Mammalian DNA”, Molecular Cloning: A Laboratory Manual Vol. 1 (third edition) (Sambrook and Russell, Cold Spring Harbor Laboratory Press, Cold Spring Harbor, N.Y., 1.31-1.38, 2001); this method, however, requires an operation using centrifugal separation. On the other hand, in recent years, techniques with which DNA extraction and purification can be performed only by adding a reagent such as Ampdirect® (manufactured by Shimadzu Corporation) have been developed. By using such techniques, DNA extraction and purification can be done regardless of the structure of the container (or cavity). In addition, DNA extraction and purification can be performed in a single cavity instead of in two separate cavities. For example, by combining the cavities 2a and 2b of FIG. 6 together into a single cavity 2ab (not shown), a card-type device having three cavities (for extraction/purification, amplification, and detection) in total may be formed.

A heat generating portion 5 having embedded therein a material, such as metal, which can generate heat by electromagnetic induction is arranged beneath the cavity 2c used for a PCR reaction, whereby only the cavity 2c can be locally heated. The PCR reaction requires a temperature rise and fall; therefore, it is important to arrange the heat generating portion 5 so that the rise and fall cycle does not influence an enzyme and the like in the cavity 2d used for detection.

In DNA detection in the cavity 2d, a method such as an optical method or electrophoresis may be used. For example, a method of measuring the concentration of a pyrophosphoric acid which is formed during DNA amplification does not involve operations such as washing and separation, and thus is useful as a detection portion of the present invention. For example, as disclosed in PCT (WO) No. 2001-506864, it is possible to use a method such as an optical method in which a pyrophosphoric acid is converted to ATP (adenosinetriphosphate) and luciferin light emission by luciferase action using the ATP as a substrate is detected, or an electrochemical detection method using pyrophosphatase, GAPDH (glyceraldehyde-3-phosphate-dehydrogenase), diaphorase, or the like. In the case of optical methods, in the card-type device, a material transparent with respect to a wavelength of light to be detected is used for either one or both of a substrate 1 above the cavity 2d and a substrate 3, and in the case of electrochemical methods, an electrode of gold, platinum, carbon, or if necessary silver/silver chloride or the like is provided in the cavity 2d.

Displacement of the sample between the cavities may be performed using electroosmotic flow, a pump, a centrifugal force, or the like. For example, it is possible to use methods such as a method (Anal. Chem., Barker et al.; (Article); 2000; 72 (24); 5925-5929) in which both positive and negative electrodes are provided and fluid is displaced using electroosmotic flow; a method (Anal. Chem., Hisamoto et al.: (Article); 2001; 73 (22); 5551-5556) in which a tube-like pipe pump is provided in a sample injection hole 4 and a pressure or suction force from the pump is used; or a method (Anal. Chem., Duffy et al.; (Article); 1999; 71 (20); 4669-4678) in which a substrate is placed on a rotatable member (e.g., a CD drive or the like) and the flow path is set in a direction in which the centrifugal force acts, whereby a sample solution is displaced using the centrifugal force.

Note that in FIG. 6, the construction is such that only one type of DNA sample is analyzed; however, a plurality of DNA may be analyzed on the chip by providing more sets of channels and cavities. In addition, needless to say, the number of sample injection holes, the number of cavities, the arrangement pattern of the cavities and channels, and the like are not limited to those described in the above-described embodiment.

The size of the card-type device of the present invention shown in FIG. 6 is not particularly limited; however, in terms of easy handling, the ideal size may be between about a slide glass size and about a credit card size (business card size). Although the volume of each cavity may vary depending on the purpose, generally, the volume may range from several nl to several ml. For example, the maximum volume of the cavity may range from about 1 nl to about 10 nl; about 10 nl to about 100 nl; about 100 nl to about 1 μl; about 1 μl to about 10 μl; about 10 μl to about 100 μl; about 100 μl to about 1 ml; or about 1 ml to about 10 ml. However, in considering the amount normally used for diagnosis purposes in hospitals or the like, the ideal volume of the cavity may range from the order of several μl to several ml. The form of the cavity is not limited to a circle and may be a polygon such as a triangle or a quadrangle.

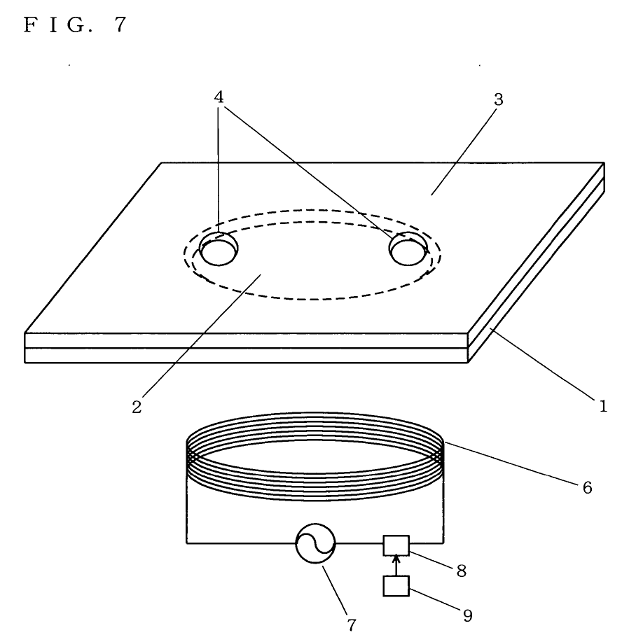

FIG. 7 is a schematic diagram showing a reaction device according to the embodiment of the present invention. The reaction device of the present embodiment includes: a reaction container as shown in FIG. 1; a heating coil 6 for heating a heat generating portion by electromagnetic induction; an alternating-current power supply 7 which acts as a power supply portion for supplying a current to the heating coil; a control portion 8 for controlling the alternating-current power supply 7; and a timer 9. Electromagnetic induction heating occurs in such a manner that the magnetic lines of force which are induced by supplying a high-frequency current to the heating coil 6 using the alternating-current power supply 7 cause the heat generating portion to generate an eddy current. The eddy current causes a loss due to the electrical resistance of the heat generating portion and the loss generates Joule heat, whereby the heat generating portion itself generates heat. The absorbed power P of the heat generating portion which is related to the heat value of the heat generating portion is expressed by the following equation:
P=KRs(NI)²
where P is the absorbed power (W) of the heat generating portion, N is the number of times the heating coil is wound, Rs is the skin resistance (Ω) of the heat generating portion, I is the current (A) of the heating coil, and K is a constant. In addition, the skin resistance Rs of the heat generating portion is expressed by:
Rs=ρ/δ
where ρ is the resistivity (Ω cm) of the heat generating portion and σ is the skin penetration depth (m) of the current.

Normally, the frequency of a high-frequency current to be supplied to the heating coil is on the order of 20-25 kHz, and thus in order to increase the heat value of the heat generating portion, the material which is included in the heat generating portion and which can generate heat by electromagnetic induction preferably has high resistivity and a low penetration depth in frequencies of 20 to 25 kHz. The preferred material includes stainless, iron, nickel, silver, or the like. In addition, an alloy, a clad metal, or the like which includes any of the above-described metals is also preferable.

Further, in recent years, a triple resonant inverter which uses a switching mode of the order of 20 kHz and converts the heating coil current frequency to about 60 kHz has been developed, and a reduction in skin effect caused by a twisted coil of a collection of low-loss fine wires has been realized, and thus nonmagnetic materials such as aluminum, copper, gold, and brass can also be preferably used.

As can be seen, the present invention is advantageous in that the selection of material for a heat generating portion is relatively flexible as compared to the case, for example, of using a resistance coil where there are limitations in the selection of material. For example, in the case of the present invention, there is an advantage in that as the material for a heat generating portion, heat-resistant materials such as SUS can be used.

By placing the reaction container on the heating coil 6 and supplying a high-frequency current to the heating coil 6, the heat generating portion generates heat by electromagnetic induction heating in accordance with the amount of the current, whereby the temperature of the sample in the cavity 2 rises. On the other hand, by stopping the supply of a high-frequency current, heat generation of the heat generating portion finishes, whereby the temperature of the sample in the cavity 2 falls. Such on/off control of the high-frequency current is performed by the control portion 8 based on the temperature measured by a temperature probe (not shown) placed on the reaction container or in the cavity, and the time measured by the timer 9. In this manner, with the use of electromagnetic induction, by adjusting the location where the heat generating portion is provided, only a desired portion to be heated is allowed to directly generate heat and thus the sample temperature rises extremely rapidly. In addition, the portion to be heated is limited to the heat generating portion, i.e., a portion of the reaction container other than where the heat generating portion is present is not heated, and therefore when heating by electromagnetic induction is stopped, the temperature falls extremely rapidly due to the heat capacity of the reaction container. Accordingly, nucleic acid amplification can be done in a shorter time compared to a nucleic acid amplification reaction using a conventional reaction container made of an aluminum block, etc. Furthermore, the form of the reaction container and the location and form of the heat generating portion can also be freely selected. In addition, since in the case of the present invention an alternating-current is used, an advantage is provided that thermal efficiency is very high and thus degradation or the like is not likely to occur.

### (EXAMPLE)

### (Example 1)

An example will be described below in which a polymerase chain reaction is performed as a nucleic acid amplification reaction, using the reaction container according to the embodiment of the present invention, as shown in FIGS. 1 and 2.

As a substrate 1, a silicon single crystal plate with a thickness of 500 μm was used and a reaction container for a polymerase chain reaction was fabricated.

First, a silicon single crystal plate with a thickness of 500 μm, the surface of which was subjected to a mirror finishing process, and a glass with a thickness of 400 μm were prepared. A cavity 2 of 6 mmø was provided in the silicon single crystal plate by dry etching using sulfur hexafluoride. The etching depth was 170 μm.

Then, two holes of 0.6 mmø, which served as sample injection holes 4, were provided by sandblasting in the glass which served as a cover plate 3.

Subsequently, the surfaces of the silicon single crystal plate and the glass were cleaned using an acid cleaner, the silicon single crystal plate and the glass were bonded together with no air therebetween, and then heated at 300° C. for three hours, whereby the substrate 1 made of a silicon single crystal plate was bonded to the cover plate 3 made of a glass by a direct bonding without using an adhesive.

Finally, the bonded substrate 1 and cover plate 3 was cut out in the form of a chip of 8 mm×16 mm and then a SUS 430 plate with a size of 8 mm×16 mm and a thickness of 1 mm was bonded as a heat generating portion 5 to substantially the entire surface of the backside, i.e., the silicon single crystal plate side, using a high-heat conductive adhesive sheet (Cerasin manufactured by Mitsubishi Gas Chemical Company, Inc.), whereby a reaction container was fabricated. In addition, as a comparative example, a 0.5 ml polypropylene tube was used as a reaction container.

In a polymerase chain reaction, λDNA (manufactured by Takara Shuzo Co., Ltd.) (for the base sequence of λDNA, see the GenBank database Accession No. V00636, J02459, M17233, X00906) was used as a template. As primers, Control Primer 1 (5′-GATGAGTTCGTGTCCGTACAACT-3′) and Primer 3 (5′-GGTTATCGAAATCAGCCACAGCGCC-3′) of TaKaRa polymerase chain reaction Amplification Kit (manufactured by Takara Shuzo Co., Ltd.) were used and an experiment was conducted (for 500 bp amplification).

After adding 0.25 μl of 2.5 U/μl TaKaRa Taq®, 5 μl of 10×PCR Buffer, 4 μl of a dNTP mixture (2.5 mM each), 2.25 μl of each 20 pmol/μl Primer 1 and Primer 3, and 2 μl of 0.25 μg/μl bovine serum albumin, 5 μl of 10 ng/μul λDNA was added and 29 μl of distilled water was added to a total volume of 50 μl.

5 μl and 20 μl of the sample prepared above were injected into the reaction container of the present example and the reaction container of the comparative example, respectively, and the sample injection holes 4 of the reaction container of the present example were sealed with a heat-resistant tape. The polymerase chain reaction using the reaction container of the comparative example was performed using an aluminum block style thermal cycler for 30 cycles each consisting of 98° C. for one minute, 55° C. for one minute, and 72° C. for 20 seconds. The total reaction time was 80 minutes. On the other hand, the polymerase chain reaction using the reaction container of the present example was performed with the reaction container placed on the heating coil, by electromagnetic induction heating, for 30 cycles each consisting of 98° C. for one second, 55° C. for one second, and 72° C. for five seconds. The temperature was measured by a thermocouple which was fixed directly on the reaction container, and controlled by on/off of a high-frequency current with a frequency of 20 kHz which was supplied to the heating coil. The total reaction time was five minutes.

After completion of the polymerase chain reaction, the container was inserted in a 1.5 ml centrifugal tube and a centrifugal process was performed at a rotational velocity of 10 krpm for one minute, and then the sample was collected from the container. The collected sample was subjected to agarose gel electrophoresis to verify whether the target 500 bp fragment had been amplified by the polymerase chain reaction. The gel used for the electrophoresis was 3% agarose. As the agarose, Agarose S manufactured by Nippon Gene Co., Ltd., was used. As the electrophoresis buffer, 50×TAE buffer (2M Tris-acetate, 50 mM EDTA) manufactured by Nippon Gene Co., Ltd., was diluted and used. The electrophoresis conditions were 100 V for 35-40 minutes.

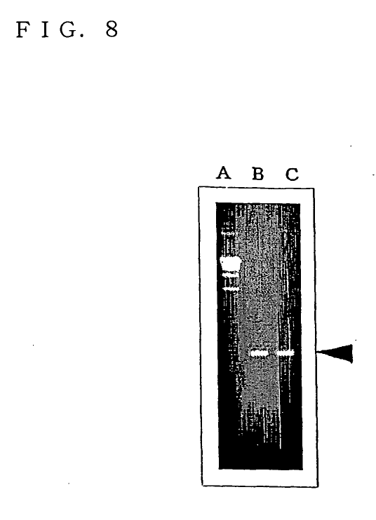

FIG. 8 is an electrophoresis picture showing the results of the polymerase chain reaction. In the drawing, A is a marker (molecular weight marker), B indicates the result obtained in the case of using the reaction container of the comparative example, and C indicates the result obtained in the case of using the reaction container of the present example. In addition, the portion indicated by the arrow indicates the target amplification products.

From FIG. 8, it can be seen that even in the case of using the reaction container of the present example, the target DNA has been amplified as is the case of using the reaction container of the comparative example. Therefore, by using the reaction container of the present example, a favorable nucleic acid amplification reaction can be obtained in one-tenth or less of the reaction time as compared to the case of using the reaction container of the comparative example.

In the case where the heating temperature and time were changed and as a comparative example, a polymerase chain reaction was performed using an aluminum block style thermal cycler for 30 cycles each consisting of 94° C. for 30 seconds, 55° C. for 30 seconds, and 72° C. for one minute, the total reaction time was 90 minutes. On the other hand, in the case where a polymerase chain reaction using the reaction container of the present invention was performed by electromagnetic induction heating for 30 cycles each consisting of 94° C. for one second, 55° C. for one second, and 72° C. for five seconds (the frequency was controlled by on/off of a high-frequency current of 20 kHz), the total reaction time was 10 minutes. In this case too, by the PCR reaction using the reaction container of the present invention, the target DNA can be amplified in the order of one-ninth of the reaction time as compared to the case of the comparative example.

Although in the present embodiment, a silicon single crystal plate and a glass were bonded together with no air therebetween and then heated at 300° C. for three hours, whereby a substrate 1 made of a silicon single crystal plate was bonded to a cover plate 3 made of a glass by a direct bonding, the temperature at the time of heating may be changed depending on the type of glass. In the case of glass containing sodium, potassium, or the like, the temperature is 250° C., and in the case of glass which does not contain such elements, the temperature is raised to the order of 400° C. In addition, for the type of glass, glass which does not contain impurities such as silica glass can also be used. In this case, the temperature is further raised to 500° C. or higher. Further, as a cover plate material, a silicon single crystal plate can also be used. In this case, a silicon single crystal plate having provided therein a cavity 2 and a silicon single crystal plate having provided therein sample injection holes 4 are bonded together by a direct bonding. The temperature at the time of bonding should be 500° C. or higher.

### (Example 2)

An example will be described below in which a polymerase chain reaction was performed as a nucleic acid amplification reaction, using a reaction container prepared in the same manner as that in example 1. As a comparative example, a 0.5 ml polypropylene tube was used as a reaction container.

In a polymerase chain reaction, a genome DNA solution which was extracted from human blood was used as a template. A DNA solution was prepared by extracting genome DNA from a subject's blood using Gen torukun™ (for blood) (manufactured by Takara Shuzo Co., Ltd.). As primers, GH20 (forward) primer (5′-GAAGAGCCAAGGACAGGTAC-3′) and GH21 (reverse) primer (5′-GGAAAATAGACCAATAGGCAG) of TaKaRa PCR control primer β-globin (human) Primer Set (manufactured by Takara Shuzo Co., Ltd.) were used and an experiment was conducted (for 408 bp amplification). After adding 0.5 μl of 2.5 U/μl TaKaRa Z-Taq®, 5 μl of 10×Z-Taq Buffer, 4 μl of a dNTP mixture (2.5 mM each), 2.25 μl of each 20 pmol/μl Primer 1 and Primer 3, and 2 μl of 0.25 μg/μl bovine serum albumin, 5 μl of 10 ng/μl human genome DNA was added and 29 μl of distilled water was added to a total volume of 50 μl.

5 μl and 20 μl of the sample prepared above were injected into the reaction container of the present example and the reaction container of the comparative example, respectively, and the sample injection holes 4 of the reaction container of the present example were sealed with a fluid sealant ELEP COAT LSS-520 (manufactured by Nitto Shinko Corporation). The polymerase chain reaction using the reaction container of the comparative example was performed using an aluminum block style thermal cycler for 30 cycles each consisting of 98° C. for five seconds and 66° C. for two seconds. The total reaction time was 20 minutes. On the other hand, the polymerase chain reaction using the reaction container of the present example was performed with the reaction container placed on the heating coil, by electromagnetic induction heating, for 30 cycles each consisting of 98° C. for two seconds and 66° C. for one second. The temperature was measured by a thermocouple which was fixed directly on the reaction container, and controlled by on/off of a high-frequency current with a frequency of 20 kHz which was supplied to the heating coil. The total reaction time was five minutes.

Further, in order to examine the influences of the form, size, heat capacity, and the like of the container with respect to the time required for one cycle of the PCR reaction, in a PCR device using a conventional aluminum block style thermal cycler, the reaction container of the present example containing a 5 μl sample was placed on the aluminum block and a PCR reaction was performed in the same manner as that in the case of using a normal polypropylene tube, without using heating by electromagnetic induction. The PCR reaction was performed for 30 cycles each consisting of 98° C. for two seconds and 66° C. for one second, and the total reaction time was 18 minutes. As can be seen, in the case of using an aluminum block style thermal cycler, even if the reaction container of the present example was used, the total reaction time took almost as much time as needed for the case of using a normal polypropylene tube. Accordingly, it has been verified that the main factor of determining the time required for one cycle in the case of using an aluminum block style thermal cycler is the heat capacity of the aluminum block itself rather than the form and the like of the container.

After completion of the polymerase chain reaction, the sample was collected in the same manner as that in example 1. The collected sample was subjected to agarose gel electrophoresis, as is the case with example 1, to verify whether the target 408 bp fragment had been amplified by the polymerase chain reaction.

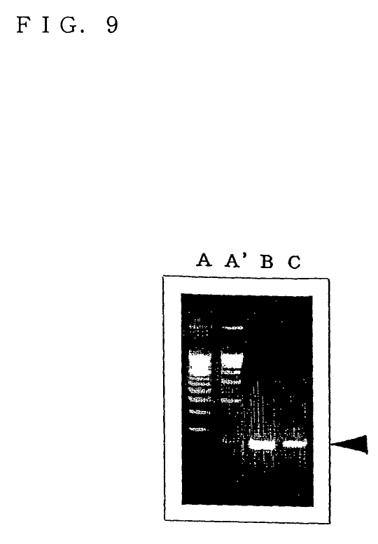

FIG. 9 is an electrophoresis picture showing the results of the polymerase chain reaction. In the drawing, A and A′ are markers (molecular weight markers), B indicates the result obtained in the case of performing PCR with an aluminum block style thermal cycler using the reaction container of the comparative example, and C indicates the result obtained in the case of performing PCR by electromagnetic induction heating using the reaction container of the present example. In addition, the portion indicated by the arrow indicates the target amplification products.

From FIG. 9, it can be seen that even in the case of using electromagnetic induction heating using the reaction container of the present example, the target DNA has been amplified as is the case of using an aluminum block style thermal cycler using the reaction container of the comparative example. Therefore, by using heating by electromagnetic induction in the reaction container of the present example, the reaction time can be reduced by a factor of about 4 as compared to the case of using an aluminum block style cycler with the reaction container of the comparative example, and also a favorable nucleic acid amplification reaction comparable to the comparative example can be obtained.

### Industrial Applicability

The reaction container, reaction device, nucleic acid detection device, and nucleic acid amplification method which use electromagnetic induction heating and which are used for a nucleic acid amplification reaction, according to the present invention are suitable for locally heating a sample and enable rapid control of temperature changes in the sample. In addition, the reaction container, reaction device, and nucleic acid detection device which are used for a nucleic acid amplification reaction, according to the present invention provide advantageous effects such as being easy to fabricate and being low cost, and are useful for applications such as diagnosis card devices for the application at the Point of Care which use nucleic acid amplification by PCR, various enzyme reactions, and the combination thereof.

1-15. (canceled)

16. A method of detecting a nucleic acid using a nucleic acid detection device and a coil provided separately from the nucleic acid detection device, the nucleic acid detection device comprising:
a substrate;
a plurality of cavities provided in the substrate; and
a cover plate adhered to the substrate, wherein
the plurality of cavities include a first cavity for amplifying a nucleic acid by a PCR reaction, and a second cavity for detecting the nucleic acid amplified in the first cavity, and
the first cavity has a heat generating portion for heating only the first cavity by electromagnetic induction, the method comprising:
a first step of injecting a sample which contains a nucleic acid into the first cavity;
a second step of amplifying the nucleic acid contained in the sample by a PCR reaction performed by supplying a current to the coil to allow the heat generating portion to generate heat by electromagnetic induction heating so that the sample injected into the first cavity is heated;
a third step of moving the sample containing the amplified nucleic acid from the first cavity to the second cavity; and
a fourth step of detecting the amplified nucleic acid contained in the sample having been moved to the second cavity.

17. The method of detecting a nucleic acid according to claim 16, wherein the heat generating portion is a conductive thin film.

18. The method of detecting a nucleic acid according to claim 16, wherein the heat generating portion is a metal thin film.

19. The method of detecting a nucleic acid according to claim 16, wherein the cavities further include a third cavity for extracting a nucleic acid, and
the method further comprises a step, prior to the first step, of extracting a nucleic acid from a sample in the third cavity and moving the extracted nucleic acid to the first cavity.

20. The method of detecting a nucleic acid according to claim 16, wherein the nucleic acid is detected using an alternating-current power supply for supplying a current to the coil.

21. The method of detecting a nucleic acid according to claim 16, wherein the cavities further have a channel for fluidly connecting the first cavity and the second cavity.

22. The method of detecting a nucleic acid according to claim 16, wherein the sample containing the nucleic acid is one selected from the group consisting of blood, saliva, and hair root.


SEQUENCE LISTING (4 sequences)

SEQ ID NO: 1 - DNA, 23 bp, Artificial Sequence
  primer 1
gatgagttcgtgtccgtacaact

SEQ ID NO: 2 - DNA, 25 bp, Artificial Sequence
  primer 3
ggttatcgaaatcagccacagcgcc

SEQ ID NO: 3 - DNA, 20 bp, Artificial Sequence
  GH20 primer
gaagagccaaggacaggtac

SEQ ID NO: 4 - DNA, 21 bp, Artificial Sequence
  GH21 primer
ggaaaatagaccaataggcag